(19) United States
(12) Patent Application Publication
(10) Pub. No.: US 2026/0207219 A1
(43) Pub. Date: Jul. 23, 2026

(54) DEVICES AND SYSTEMS FOR THROMBUS TREATMENT FIELD

(71) Applicant: W. L. Gore & Associates, Inc., Newark, DE (US)

(72) Inventors: Mark C. Bates, Encinitas, CA (US); Edward E. Shaw, Flagstaff, AZ (US)

(21) Appl. No.: 19/139,697

(22) PCT Filed: Dec. 15, 2023
(86) PCT No.: PCT/US2023/084210

§ 371 Date: Jun. 16, 2025

Related U.S. Application Data
(60) Provisional application No. 63/432,886, filed on Dec. 15, 2022.

(51) Int. Cl. A61B 17/3207; A61B 17/22

(52) U.S. Cl. CPC A61B 17/320725; A61B 2017/22084; A61B 2017/320716

(57) ABSTRACT

A medical device (100) for treating a thrombus or embolism that includes a first elongate element (110) having a first end and a second end, defines a lumen, and has a first diameter, a second elongate element (120) extending through at least a portion of the lumen of the first elongate element, the second elongate element having a first end and a second end, wherein the first end of the second elongate element is configured to extend beyond the first end of the first elongate element, a frame (132) extending from the first end of the second elongate element, the frame having a second diameter in a deployed configuration, wherein the second diameter is greater than the first diameter of the first elongate element, and a plurality of lines (150) coupled to the first elongate element and the frame, the plurality of lines operable to be tensioned when the frame is in the deployed configuration.

### CROSS-REFERENCE TO RELATED APPLICATION

This application is a national phase application of PCT Application No. PCT/US 2023/084210, internationally filed on Dec. 15, 2023, which claims the benefit of Provisional Application No. 63/432,886, filed Dec. 15, 2022, which are incorporated herein by reference in their entireties for all purposes.

### FIELD

The present disclosure relates generally to apparatuses, systems, and methods treating thrombosis.

### BACKGROUND

Interruption of venous or arterial flow from blood clot formation within the vessel (“thrombosis), or blood flow directed egress of blood clot from one location to another (embolism) can result in serious life-threatening consequences such as ischemic stroke, acute limb ischemia, myocardial infarction (heart attack), pulmonary embolism, and deep vein thrombosis (DVT). Occlusive blood clots, or “thrombi”, within arteries can acutely deprive tissue and organs of oxygen while venous thrombotic occlusion can cause progressive swelling with acute and long-term tissue injury risks. One of the most devastating examples of embolism is ischemic stroke in which blood flow obstruction to the brain is sustained for longer than a few seconds and which can result in irrecoverable brain cell death and permanent neurological damage.

Thrombi can be treated (reduced or eliminated) by inducing thrombolysis. Thrombolysis is the dissolving, or “lysis,” of a thrombus. Thrombolysis can sometimes be induced pharmacologically, such as by administering a tissue plasminogen activator drug (tPA), the most common thrombolytic agent. Thrombolytic agents (commonly called “clot-busting drugs”) can be administered via an intravenous line or using a catheter to deliver them proximally to the thrombus. However, thrombolysis by administration of clot-busting drugs has its limitations. For example, to be successful, the clot-busting drugs should be administered within three (3) hours of an acute ischemic stroke, and preferably within two (2) hours. Further, patients who use blood-thinning medications, and certain other medications, are usually not candidates for pharmacological thrombolysis. And of those patients receiving the treatment, it is unsuccessful in dissolving thrombi in approximately 25% of patients. Additionally, there is a significant risk of bleeding with thrombolysis and thus it is no longer the preferred treatment for acute myocardial infarction and reserved in pulmonary embolism only for those patients in shock with otherwise a very poor prognosis, as examples.

In view of the limitations of pharmacologically induced thrombolysis, various medical devices for catheter enabled (“endovascular”) removal of thrombi have been developed. The procedure for endovascular removal of thrombi is generally known as “transcatheter thrombectomy.” In thrombectomy treatments, a catheter system is typically used to deliver a device to the thrombus. The device can be, for example, an aspiration catheter. Aspiration catheters can perform a thrombectomy by suctioning the thrombus out of the blood vessel. Other thrombectomy procedures use a mechanical device to physically entangle with a thrombus, and to remove the thrombus as the device is removed from the blood vessel with or without the enablement of a suction catheter. Various types of mechanical devices, such as wires, corkscrew-like coils, bristles, and baskets have been employed to entangle with thrombi.

Some traditional thrombectomy devices can cause damage to blood vessel walls. In addition, some traditional thrombectomy devices can be prone to generating thrombotic fragments that become emboli when they travel within the bloodstream. Emboli can become lodged in arteries, veins, arterioles, and capillaries, and can block the blood supply to vital organs such as the brain, lungs, or heart. Emboli in the bloodstream can be life-threatening. In the case of DVT treatment, dislodged thromboemboli can travel to the lungs, resulting in a pulmonary embolism, which can be fatal.

### SUMMARY

This specification describes devices, systems, and processes for treatment of thrombi. In brief, various embodiments are disclosed for mechanically restoring a blood-flow path, facilitating lysis by blood flow, withdrawing thrombotic material, and capturing thrombotic fragments in a filter device. Additionally, devices, systems, and processes for maceration, aspiration and other adjunct processes are disclosed.

According to one example (“Example 1”), a medical device for treating one of a thrombus and embolism includes a first elongate element having a first end and a second end, the first elongate element defining a lumen and having a first diameter; a second elongate element extending through at least a portion of the lumen of the first elongate element, the second elongate element having a first end and a second end, wherein the first end of the second elongate element is configured to extend beyond the first end of the first elongate element; a frame extending from the first end of the second elongate element, the frame having a second diameter when in a deployed configuration, wherein the second diameter is greater than the first diameter of the first elongate element; and a plurality of lines coupled to the first elongate element and the frame, the plurality of lines operable to be tensioned when the frame is in the deployed configuration, the plurality of lines operable to cut through at least one of an embolism or thrombus.

According to another example (“Example 2”), further to Example 1, the medical device further includes a vacuum source, wherein the lumen of the first elongate element is operable to be fluidically coupled to the vacuum source.

According to another example (“Example 3”), further to Example 1, the first elongate element includes sufficient structural integrity to support being advanced through the embolism or thrombus.

According to another example (“Example 4”), further to Example 1, the second elongate element is longitudinally moveable relative to the first elongate element.

According to another example (“Example 5”), further to Example 1, the second elongate element includes a lumen operable to accommodate a guidewire.

According to another example (“Example 6”), further to Example 5, the second elongate element includes at least one opening proximate the first end, the second elongate element is operable to be fluidically coupled to a fluid source including at least one of a contrast source, a saline source, and a therapeutic source. The contrast and saline can be actively or passively released during suction in a way that allows direct visualization of thrombus behavior with reduction of blood loss and simultaneous removal of the contrast to reduce contrast exposure for the patient.

According to another example (“Example 7”), further to Example 1, the frame includes a nitinol structure operable to at least partially self-expand when transitioned from a delivery configuration to a deployed configuration.

According to another example (“Example 8”), further to Example 1, the frame includes a porous covering.

According to another example (“Example 9”), further to Example 1, the frame defines an outer perimeter, wherein each line of the plurality of lines is coupled to the frame proximate the outer perimeter.

According to another example (“Example 10”), further to Example 9, the frame includes a plurality of radial struts extending from the second elongate element toward the outer perimeter of the frame.

According to another example (“Example 11”), further to Example 10, each line of the plurality of lines is coupled to the frame at the plurality of radial struts.

According to another example (“Example 12”), further to Example 9, the frame includes a plurality of peripheral struts extending between the plurality of radial struts proximate the outer perimeter of the frame, wherein the plurality of lines are coupled to the frame at the plurality of peripheral struts between the plurality of radial struts.

According to another example (“Example 13”), further to Example 1, each line of the plurality of lines includes a resilient polymer or composite e.g., an ePTFE filament.

According to another example (“Example 14”), further to Example 1, each line of the plurality of lines is a braided fiber.

According to another example (“Example 15”), further to Example 1, the plurality of lines is coupled to an outer surface of the first elongate element.

According to another example (“Example 16”), further to Example 1, the medical device further comprises a secondary cutter coupled to the second elongate element proximate the first end of the first elongate element.

According to another example (“Example 17”), further to Example 1, the medical device further comprises an expandable member coupled to the first elongate element.

According to another example (“Example 18”), further to Example 17, the expandable member is positioned interior to the plurality of lines such that when the expandable member is expanded, the plurality of lines are tensioned.

According to another example (“Example 19”), further to Example 1, the second elongate element is rotatable relative to the first elongate element, wherein the plurality of lines are operable to be tensioned when the second elongate element is rotated.

According to another example (“Example 20”), further to Example 1, the plurality of lines is coupled to an outer surface of the second elongate element.

According to one example (“Example 21”), a method of performing a thrombectomy includes advancing a medical device toward a thrombus, the medical device including a first elongate element having a first end and a second end, the first elongate element defining a lumen and having a first diameter, a second elongate element extending through at least a portion of the lumen of the first elongate element, the second elongate element having a first end and a second end, wherein the first end of the second elongate element is configured to extend beyond the first end of the first elongate element, a frame extending from the first end of the second elongate element, the frame having a second diameter when in a deployed configuration, wherein the second diameter is greater than the first diameter of the first elongate element, and a plurality of lines coupled to the first elongate element and the frame, the plurality of lines operable to be tensioned when the frame is in the deployed configuration; extending the medical device through the thrombus such that the frame and plurality of lines are positioned beyond the thrombus; deploying the frame to the deployed configuration such that the plurality of lines are tensioned; and translating the medical device longitudinally such that the lines cut through the thrombus.

According to another example (“Example 22”), further to Example 21, the method further comprises rotating the second elongate element relative to the first elongate element.

According to another example (“Example 23”), further to Example 21, the method further comprises providing contrast to proximate the thrombus through a second lumen of the second elongate element.

According to another example (“Example 24”), further to Example 21, the method further comprises suctioning through a first lumen of the first elongate element portions of the thrombus that are cut.

According to one example (“Example 25”), a medical device for treating at least one of a thrombus and embolism includes an elongate element having a first end and a second end, the first elongate element defining a lumen and having a first diameter, the first end including an opening to the lumen; a plurality of lines coupled to the first elongate element such that the lines are extending across the opening, the plurality of lines operable to be tensioned when the elongate element is in a deployed configuration, the plurality of lines operable to cut through at least one of an embolism or thrombus.

According to one example (“Example 26”), a medical device for treating at least one of a thrombus and embolism includes a first elongate element having a first end and a second end, the first elongate element defining a lumen and having a first diameter, the first end including an opening to the lumen; a frame coupled to the first end of the first elongate element, the frame having a delivery configuration and a deployed configuration; a second elongate element having a first end and a second end, the second elongate element being positioned at least partially within the lumen of the first elongate element and extending away from the first end of the first elongate element; a plurality of lines coupled to the first elongate element and the second elongate element, the plurality of lines operable to be tensioned when the frame is in the deployed configuration, the plurality of lines operable to cut through at least one of an embolism or thrombus.

According to one example (“Example 27”), a medical device for treating at least one of a thrombus and embolism includes an elongate element having a first end and a second end, the first elongate element defining a lumen and having a first diameter, the first end including an opening to the lumen; a braided frame having a delivery configuration and a deployed configuration; a plurality of lines between the elongate element and the braided frame such, the plurality of lines operable to be tensioned when the braided frame is in the deployed configuration, the plurality of lines operable to cut through at least one of an embolism or thrombus.

According to one example (“Example 28”), a medical device for treating at least one of a thrombus and embolism includes a first elongate element having a first end and a second end, the first elongate element defining a lumen and having a first diameter, the first end including an opening to the lumen; a second elongate element extending through at least a portion of the lumen of the first elongate element, the second elongate element having a first end and a second end, wherein the first end of the second elongate element is configured to extend beyond the first end of the first elongate element; an expandable member positioned about the second elongate element; and a plurality of lines extending between the second elongate element and the expandable element, the plurality of lines operable to be tensioned when the expandable element is in a deployed configuration, the plurality of lines operable to cut through at least one of an embolism or thrombus.

According to another example (“Example 29”), further to Example 28, the plurality of lines extend from the expandable member proximally and distally to second elongate element.

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure:

FIG. 1 illustrates a device for performing a thrombectomy, in accordance with an embodiment;

FIGS. 2A and 2B illustrate a device for performing a thrombectomy in a deployed and a constrained configuration, respectively, in accordance with an embodiment;

FIGS. 3-5 are a series of illustrations depicting an example manner of use of a thrombectomy device, in accordance with an embodiment;

FIG. 6 illustrates a device for performing a thrombectomy including a membrane and fluidically coupled to a suction source and a fluid source, in accordance with an embodiment;

FIG. 7 illustrates a device for performing a thrombectomy with cutting lines coupled to an inner elongate member, in accordance with an embodiment;

FIG. 8 illustrates a device for performing a thrombectomy with a frame and with cutting lines on a forward facing position, in accordance with an embodiment;

FIG. 9 illustrates a device for performing a thrombectomy with cutting lines on a forward facing position, in accordance with an embodiment;

FIG. 10 illustrates a device for performing a thrombectomy with cutting lines coupled between a first elongate element that is fluted and a second elongate element, in accordance with an embodiment;

FIGS. 11 and 12 illustrate devices for performing a thrombectomy with cutting lines coupled to a braided frame, in accordance with an embodiment;

FIG. 13 illustrates a device for performing a thrombectomy with a secondary cutter, in accordance with an embodiment;

FIG. 14 illustrates a device for performing a thrombectomy with an occluding balloon, in accordance with an embodiment;

FIG. 15 illustrates a device for performing a thrombectomy with a second elongate element that may be rotated to tension lines for cutting, in accordance with an embodiment;

FIG. 16 illustrates a device for performing a thrombectomy with a frame with rotatable struts for cutting, in accordance with an embodiment;

FIG. 17 illustrates a device for performing an antegrade thrombectomy with a balloon for transitioning lines for cutting to a deployed configuration, in accordance with an embodiment;

FIG. 18 illustrates a device for performing a retrograde thrombectomy with a balloon for transitioning lines for cutting to a deployed configuration, in accordance with an embodiment; and

FIG. 19 illustrates a device for performing an antegrade and a retrograde thrombectomy with a balloon for transitioning lines for cutting to a deployed configuration, in accordance with an embodiment.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

### Description of Various Embodiments

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

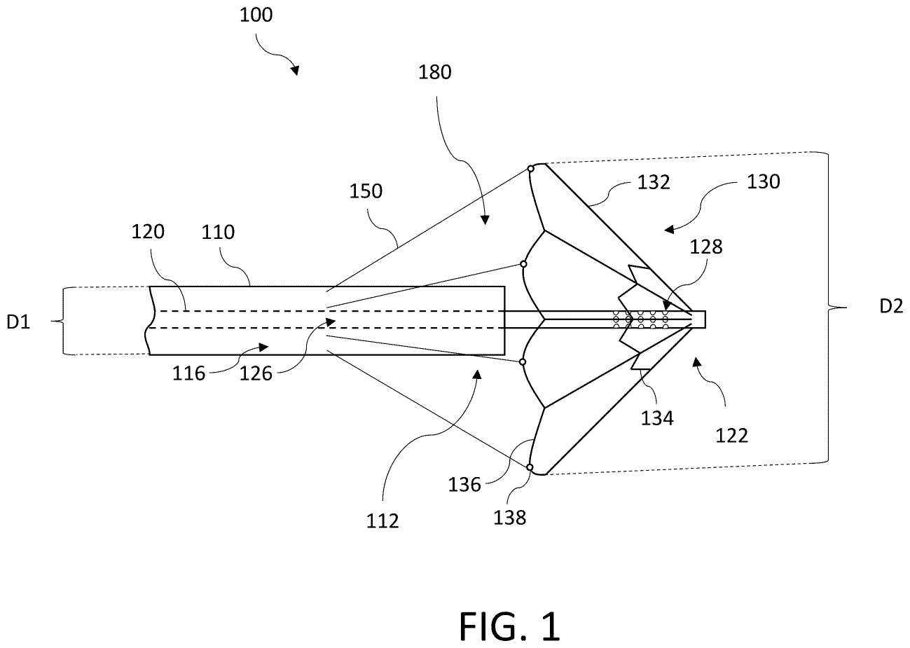

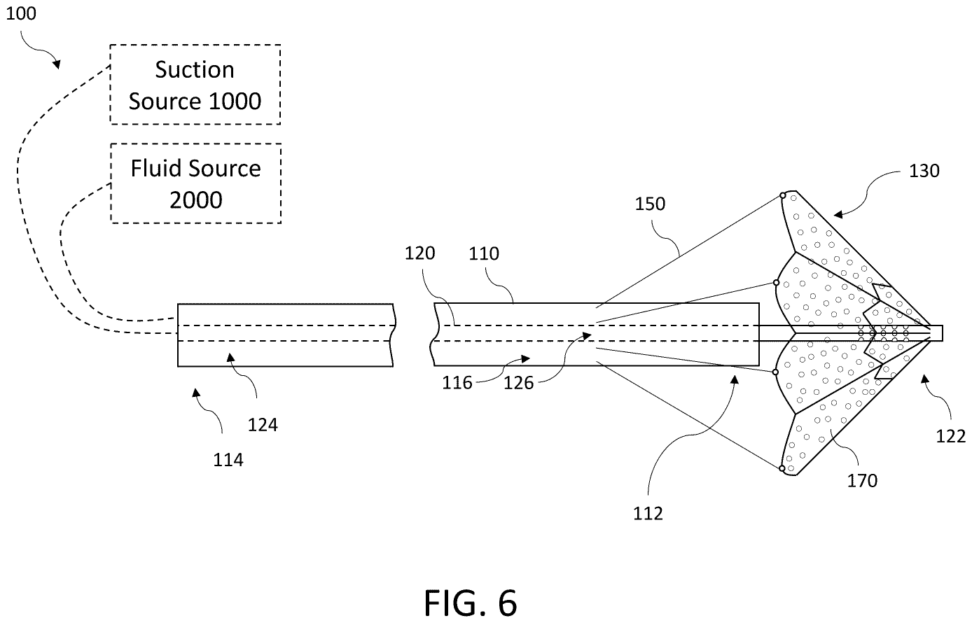

The thrombectomy device shown in FIG. 1 is provided as an example of the various features of the device and, although the combination of those illustrated features is clearly within the scope of invention, that example and its illustration is not meant to suggest the inventive concepts provided herein are limited from fewer features, additional features, or alternative features to one or more of those features shown in FIG. 1. For example, in various embodiments, the thrombectomy device shown in FIG. 1 may include the filter membrane described with reference to FIG. 6. It should also be understood that the reverse is true as well. One or more of the components depicted in FIG. 1 can be employed in addition to, or as an alternative to components depicted in other Figures.

The disclosure relates to a medical device for performing a thrombectomy and is illustrated herein. The medical device is capable of breaking up and removing a vascular defects or vascular occlusions such as a thrombus or embolism (e.g., blood clot or plaque)from the lumen of a patient. The medical device is also capable of limiting emboli from travelling through the vasculature after the embolism is broken up. The medical device is also capable of atraumatically deploying and breaking up the embolism within the vasculature of the patient. The medical device 100 is also capable of providing visualization and/or treatment to the thrombus during removal. The medical device is also capable of actively and/or passively capturing the broken-up portions of the thrombus.

Referring to FIG. 1, a medical device 100 is illustrated. The medical device 100 includes a first elongate element 110 having a first end 112 and a second end 114, the first elongate element 110 defining a lumen 116 and having a first diameter D1. The medical device 100 further includes a second elongate element 120. The second elongate element 120 extends through at least a portion of the lumen 116 of the first elongate element 110. The second elongate element 120 having a first end 122 and a second end 124. The first end 122 of the second elongate element 120 is configured to extend beyond the first end 112 of the first elongate element 110. The medical device 100 includes a frame 130 extending from the first end 122 of the second elongate element 120. The frame 130 includes a second diameter D2 when in a deployed configuration, wherein the second diameter D2 is greater than the first diameter D1 of the first elongate element 110. The medical device 100 further includes a plurality of lines 150 coupled to the first elongate element 110 and the frame 130, the plurality of lines 150 operable to be tensioned when the frame 130 is in the deployed configuration. The plurality of lines 150 is operable to cut through one of an embolism or thrombus.

With further reference to FIG. 1, the first elongate element 110 may be a catheter or sheath that is capable of being advanced through the vasculature of the patient. The first elongate element 110 may be manipulated by a user (e.g., a surgeon) from outside the body of the patient while the first end 112 is advanced toward and positioned proximate the thrombus within the vasculature of the patient. In some embodiments, the first elongate element 110 is in fluid communication with a suction source 1000 (e.g., a vacuum, see FIG. 6) to provide active suction for removing the thrombus. The lumen 116 of the first elongate element 110 is thus capable of providing a passage by which the thrombus is removed when the lumen is fluidically coupled to the suction source 1000. In some embodiments, the first elongate element 110 is capable of passive thrombus removal/transport. The first elongate element 110 may be provided in various diameters and lengths. The diameter of the first elongate element 110 is selected such that the first elongate element 110 is capable of being positioned and moved within the vasculature of the patient. Furthermore, the diameter of the first elongate element 110 may be sufficient to accommodate removal of the thrombus through the lumen 116 of the first elongate element 110 (e.g., cut-up portions of the thrombus). The first elongate element 110 further includes sufficient structural integrity (e.g., columnar strength) to be advanced within the vasculature of the patient and through at least a portion of the thrombus.

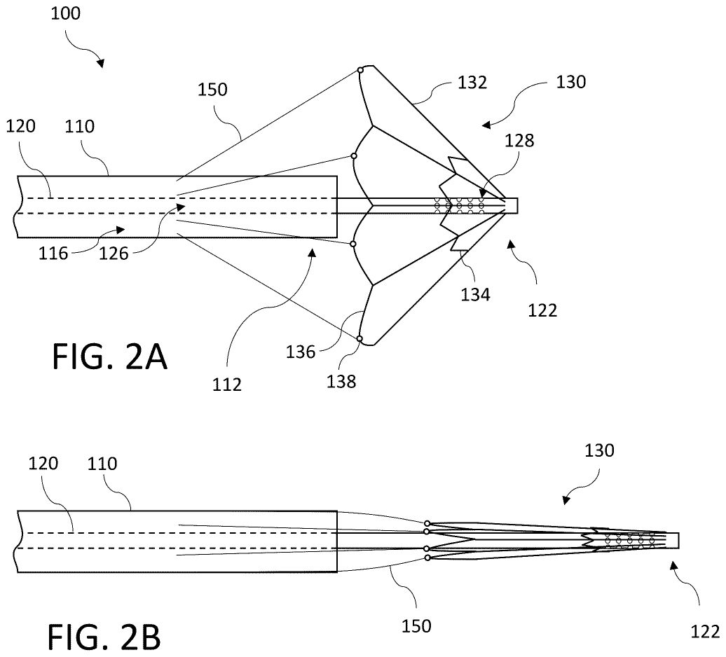

With further reference to FIG. 1, the second elongate element 120 is positioned extending through at least a portion of the lumen 116 of the first elongate element 110. The second elongate element 120 is sized such that it can be accommodated within the lumen 116 of the first elongate element 110 and allows sufficient space for portions of the thrombus to be accommodated and/or transported through the lumen 116 of the first elongate element 110. The second elongate element 120 may be longitudinally moveable within the first elongate element 110, which allows the second elongate element 120 to be advanced or retracted within the lumen 116 of the first elongate element 110. Advancing of the second elongate element 120 within the lumen 116 of the first elongate element 110 is illustrated in FIGS. 2A and 2B. By advancing and retracting (e.g., telescopically) the second elongate element 120, the device 100 can be reconfigured between a delivery configuration, a deployed configuration, and a retrieval or removal configuration. It is understood that in some embodiments, a constraint (not shown) may be implemented to constrain the medical device 100 to a delivery configuration during delivery of the medical device 100 to the target site.

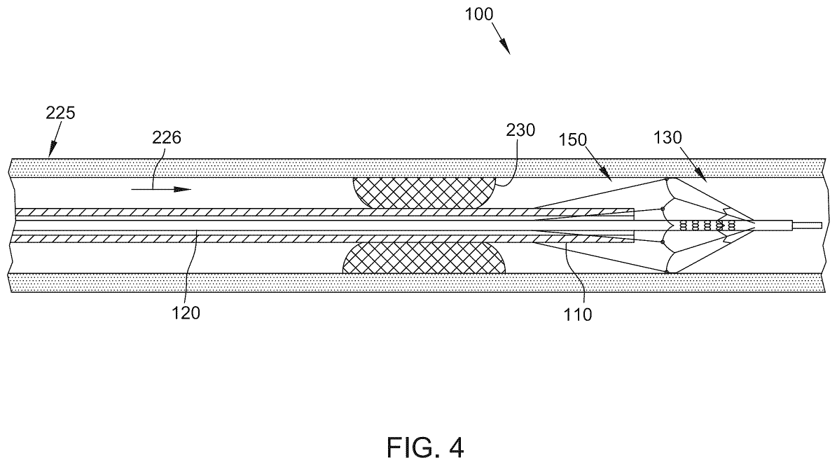

In some embodiments, the second elongate element 120 includes a lumen 126 operable to accommodate a guidewire 160 (see FIG. 4). The medical device 100 can be advanced to the target site by tracking over the guidewire 160. The lumen 126 of the second elongate element 120 may also be operable to deliver a fluid to the target site. For example, in some embodiments, the lumen 126 is fluidically coupled to a fluid source 2000 (see FIG. 6). The fluid source 2000 may include various fluids or combinations of fluids. For example, the fluid source may include a contrast fluid configured to be implemented in visualization of the target site, a saline (e.g., to replace fluids that might be removed during suction), or a therapeutic operable to reduce coagulation and/or break up the clot. It is understood that any fluid useful may be delivered at the target site via the lumen 126 of the second elongate element 120. The fluid source 200 allows for active injections and/or passive suction-enhanced flow.

In some embodiments, the second elongate element 120 includes at least one opening 128 proximate the first end 122. The opening 128 may be at a longitudinal end (e.g., through which the guidewire 160 extends), or the opening 128 may be positioned through the side walls of the second elongate element 120. For example, as illustrated in FIG. 1, the second elongate element 120 includes a plurality of openings 128 at the first end 122 of the device through the side wall (positioned circumferentially and longitudinally spaced about the first end 122 of the second elongate element 120). The opening 128 allows the fluid to be delivered at the first end 122 of the second elongate element 120, which is positioned at the target site, and the fluid may be deployed along the longitudinal axis or radially outward from the second elongate element 120.

Referring still to FIG. 1, the frame 130 extends longitudinally outward from the second elongate element 120. The frame 130 is operable to be positioned in a delivery configuration (e.g., collapsed configuration, see FIG. 2B) and a deployed configuration (e.g., expanded configuration, see FIG. 2A). The frame 130 includes a plurality of main struts 132 (e.g., radial struts) extending from the second elongate element 120. The main struts 132 may be spaced circumferentially about the second elongate element 120. The frame 130 may further include support struts 134 that interconnect the main struts 132 in order to provide structural support to the main struts 132. The support struts 134 may be provided in various configurations and at various positions along the main struts 132. As illustrated, one ring of support struts 134 may be provided, however, any number of rings of support struts 134 may be provided.

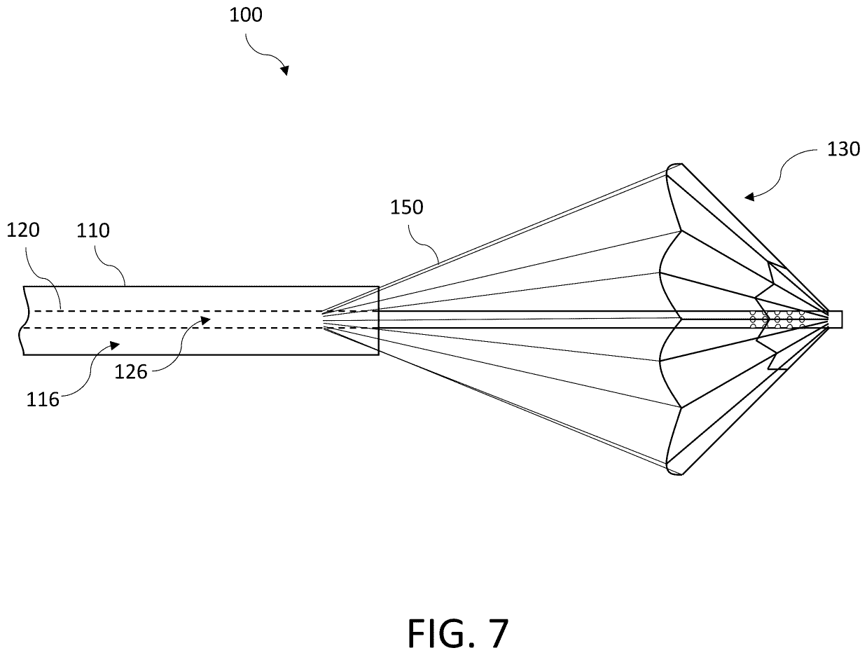

The frame 130 may further include peripheral struts 136. The peripheral struts 136 interconnect the ends of the main struts 132. The peripheral struts 136 define the outer periphery or circumference of the frame 130. The peripheral struts 136 are operable to contact the tissue (e.g., vessel walls) when in the deployed configuration. The peripheral struts 136 may be provided in various configurations, including but not limited to an arcuate shape as illustrated in FIG. 1. The arcuate shape of the peripheral struts 136 may provide an atraumatic surface for contacting the tissue so as to limit disruption of the tissue during performance of the procedure. In some embodiments, the frame 130 includes coupling positions 138 to which the plurality of lines 150 are operable to couple. In some embodiments, the coupling positions 138 may include eyelets through which the lines 150 may extend. In other embodiments, the coupling positions 138 are defined on the frame 130 and are not a specific structure integral with the structure of the frame 130. The coupling position 138, in some embodiments, may be along the peripheral struts 136. In other embodiments, the coupling position 138 may be at an intersection of the peripheral struts 136 and the main struts 132 (See FIG. 7). The coupling of the lines 150 to the frame 130 may be provided in various ways, including but not limited to, knots, adhesives, bonding, and so forth. In some embodiments, the lines 150 may be coupled to the coupling positions 138 by looping the lines through eyelets. This also allows for the lines 150 to be dual or parallel at each coupling position 138.

The frame 130 may be formed of various materials, including but not limited to nitinol (NiTi). In addition or alternatively, other materials such as, but not limited to, stainless steel, L605 steel, polymers, MP35N steel, polymeric materials, Pyhnox, Elgiloy, or any other appropriate biocompatible material, and combinations thereof, can be used as the material of the frame 130. The super-elastic properties and softness of NiTi may enhance the conformability of the frame 130. In addition, NiTi can be shape-set into a desired shape. That is, NiTi can be shape-set so that the frame tends to self-expand into a desired shape when the frame 130 is unconstrained, such as when the frame 130 is deployed. In some embodiments, the frame 130 may additionally or alternatively be deployed via a push and pull deployment method. For example, the frame 130 is configured to be controllable throughout a range of various degrees of expansion such that the outer diameter is controllable to various sizes, or diameters. The frame 130 is controllable to a collapsed configuration with an outer diameter less than the inner diameter of the first elongate element 110. By advancing the second elongate element 120 longitudinally out of the first elongate element 110, the lines 150 are tensioned and the frame 130 may be collapsed inward. In some embodiments, a deployment mechanism may be provided.

The frame 130 is configured to be movable relative to the first elongate element 110 along the central longitudinal axis. The frame 130 may be received at least partially within the first elongate element 110, for example, by advancing the first end 112 of the first elongate element 110 over at least a portion of the frame 130. That is, in the collapsed configuration the frame 130 is at least partially slidably received within the first elongate element 110 and extendable therefrom. In some implementations, the frame 130 is self-expanding as the frame 130 is extended from the first elongate element 110.

The frame 130 is structurally or materially configured such that at least some continuous flow is maintained through a vessel when deployed (e.g., with only minor disruption to flow). The struts 132, 134, 136 may separate from each other, creating space between the struts 132, 134, 136 as the frame 130 outwardly extends. The struts 132, 134, 136 may be formed to define an open interior structure bounded by a mesh-like structure or a ribbed structure (e.g., membrane 170), for example. The spaces between the struts 132, 134, 136 may be generally longitudinally oriented (e.g., along the central longitudinal axis) or have other configurations (e.g., diagonal, helical, or others).

As shown in FIG. 1, in the deployed configuration the struts 132, 134, 136 of the frame 130 are spaced apart to define spaces between the struts 132, 134, 136 such that fluid is allowed to flow around the first elongate element 110 and through the spaces between the struts 132, 134, 136 of the frame 130. The frame 130 has gaps between struts 132, 134, 136 and defines a plurality of open spaces between struts 132, 134, 136 and an open interior space through which fluid can flow when the frame 130 is in the expanded configuration. In some examples, fluid can also flow through the spaces between the struts 132, 134, 136 when the frame 130 is being transitioned between the collapsed configuration and the expanded configuration.

Among other advantages, continuous perfusion of downstream body systems can also be beneficial. As another example, maintaining downstream perfusion can help allow for positional accuracy of the device 100 during a procedure within a vessel, because allowing continuous fluid flow reduces the need to withstand or resist pressures associated with temporary occlusion of the vessel. In other contexts, it may be desirable to block, or partially block fluid flow when the device 100 is deployed to the expanded state. In such instances, the struts 132, 134, 136 may include a membrane 170 (e.g., occluding or filtering) (see FIG. 6) extending between the struts 132, 134, 136 to block or filter the spaces such that fluids can flow through, but particulate matter over a specific size is captured by the membrane 170. The membrane 170, for example, may be a porous covering with pores to facilitate the filtering and perfusion discussed herein.

Referring still to FIG. 1, the frame 130 defines an outer perimeter that is provided, for example, by the peripheral struts 136. The plurality of lines 150 are coupled to the frame at or proximate the outer perimeter such that, when the frame 130 is in the deployed configuration, that lines 150 extend substantially toward the vessel wall. For example, the lines 150 may be coupled to the peripheral struts 136 or the lines may be coupled to the main struts 132 proximate the outer perimeter of the frame 130. In some embodiments, the lines 150 are formed of a suture line. For example, the lines 150 may be formed of expanded polytetrafluoroethylene (ePTFE) filaments. In some embodiments, the lines 150 are formed of braided fibers. In some embodiments, the lines may be coated or otherwise contain a drug that may aid in cutting (e.g., an anticoagulant)

In some embodiments, the plurality of lines 150 are coupled to an outer surface of the first elongate element 110. By coupling the lines 150 to the first elongate element 110, an interior space 180 is defined between the lines 150 and the frame 130. The lumen 116 of the first elongate element 110 is in fluid communication with the interior space 180 such that any embolism that is cut by the lines 150 and passes into the interior space 180 does not have to pass through another barrier (e.g., pass through the lines 150 a second time) in order to be removed through the lumen 116 of the first elongate element 110. Referring to FIG. 7, in some embodiments, the lines 150 are coupled to an exterior surface of the second elongate element 120.

Referring to FIGS. 2A and 2B, the frame 130 may be transitioned between a deployed configuration (FIG. 2A) and a collapsed configuration (FIG. 2B). The device 100 may be transitioned to the collapsed configuration to capture and remove portions of the embolism as well as for repositioning within and/or removal from the vessel.

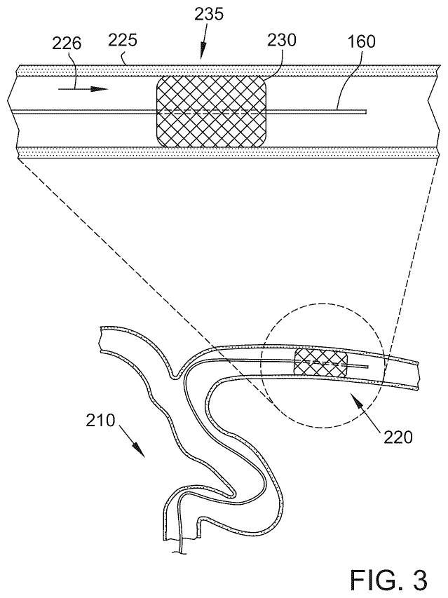

FIGS. 3-5 illustrate example devices, systems, and processes for treatment of thrombi. In general, the embodiments and concepts described can be applied in virtually any vascular region containing thrombi, for example, neurovascular, cardiovascular, and peripheral vessels, and in both arterial and venous vasculature systems. The embodiments and concepts described generally pertain to: (1) opening a blood-flow path through a vessel obstructed by a thrombus and (2) capturing and removing an amount of thrombotic material.

FIG. 3 illustrates an example vasculature portion 210 including a thrombus 230 at a thrombus site 235. The thrombus 230 can be, for example, attached to or lodged against a vessel wall 225, or lodged within a vessel 220. The thrombus 230 can partially or completely block the blood flow 226 through vessel 220. While the example of FIG. 3 depicts a thrombus 230 that partially blocks blood flow 226 through vessel 220, the devices and techniques described herein may also be used for clots or thrombi that completely block blood flow through a vessel.

Typically, access to the thrombus 230 can be initially achieved by a flexible guidewire 160. In some cases, other devices such as one or more guide catheters (not shown) may also be used to navigate through the patient's vasculature to a location near a target thrombus. In some cases, access to the thrombus can be achieved by the combination of one or more guide catheters and guidewires. For example, a combination of successively smaller guide catheters can be arranged in a telescope-like fashion. In some implementations, guidewire 160 can be inserted in vessel 220 so that the distal tip of guidewire 160 extends past the thrombus site 235.

In some embodiments, the guidewire 160 is removed from the first elongate element 110. In this embodiment, the guidewire 160 aided the navigation of the first elongate element 110 to a desired position. With the first elongate element 110 in the desired position, the guidewire 160 can be removed to make room within the lumen of first elongate element 110 for insertion of other devices to treat the thrombus 230, and other vessel obstructions or conditions, according to some implementations. In some embodiments, the guidewire 160 is left in place, whereby the guidewire 160 can be used to facilitate additional deployment operations.

While FIGS. 3-5 depict an implementation in which the target thrombus 230 is generally concentric with the vessel wall 225, in some implementations a thrombus is eccentrically positioned within a vessel. That is, the location of the thrombus may be biased to a particular side of the vessel. In such implementations, the device 100 can be inserted around (rather than through) the thrombus. However, the principles of operation of the device 100 in the context of an eccentrically positioned thrombus are generally the same as described herein in relation to the concentric thrombus of FIGS. 3-5.

FIG. 4 illustrates a device 100 in an expanded (e.g., deployed) configuration proximate a thrombus 230. In some implementations, this arrangement can be achieved by extending the first elongate element 110, while maintaining or restraining the second elongate element 120 in its prior axial position with respect to the thrombus 230. The second elongate element 120 may also be retracted while maintaining or restraining the first elongate element 110 in its prior axial position with respect to the thrombus 230.

As described previously, the frame 130 of the device 100 can, in some embodiments, be self-expanding. That is, the frame 130 can have a shape-memory characteristic that urges the frame to assume an expanded configuration (refer to FIG. 1) when it is unconstrained (e.g., unconstrained after emerging from a delivery catheter). In some embodiments, the frame 130 may assume a partially expanded configuration when it is partially constrained (as by thrombus 230 or the vessel wall 225). The device 100 will expand such that the frame 130 (e.g., the peripheral struts 136) will substantially make contact with the inner vessel wall 225. In those embodiments including a membrane 170, the membrane 170 will substantially make contact with the inner vessel wall 225. In that manner, one or more dislodged thrombotic fragments separated from the thrombus 230 by deployment of the frame 130 can be captured by the membrane 170.

Once the frame 130 is expanded, the frame 130 is positioned against the vessel wall 225. The frame 130 and the lines 150 are positioned on the distal side of the thrombus 230 such that the frame 130 is in the vessel on a side opposite from the first elongate element 110. The lines 150 are tensioned by positioning the second elongate element 120 relative to the first elongate element 110 such that the frame 130 is capable of seating within the vessel while still maintaining tension on the lines 150 between the frame 130 and the first elongate element 110. Because the lines 150 are tensioned, the lines 150 are capable of cutting through a portion of the thrombus 230 in order to break up the thrombus into smaller pieces. This can be accomplished by pulling the device 100 proximally such that the lines 150 and the frame 130 are pulled through the position where the thrombus resides. For example, the device 100 is pulled retrograde relative to the flow (e.g., blood flow) through the vessel. As the lines 150 are pulled through the thrombus 230, the thrombus 230 is broken up into smaller pieces.

As the thrombus 230 is broken up, it is possible that portions of the thrombus 230 become loose and could embolize. The frame is capable of capturing embolisms as the blood continues to flow downstream. In those embodiments including a membrane 170, the membrane 170 is further capable of capturing embolisms as the blood flows through the membrane 170. In some embodiments, the membrane 170 may be provided with a hydrophobic coating.

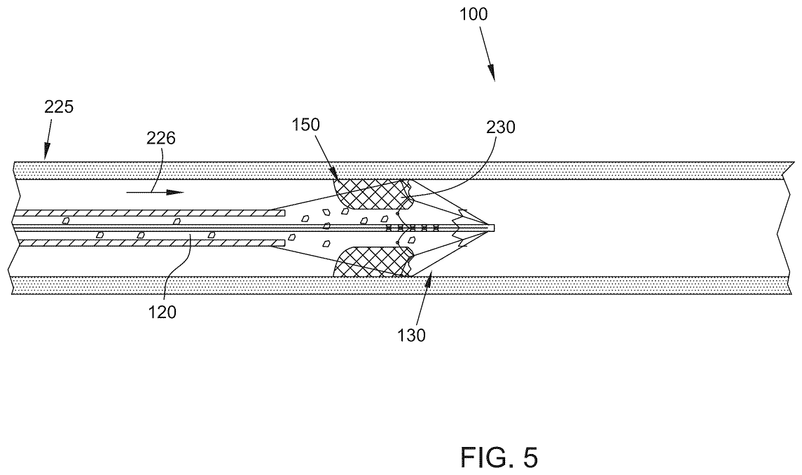

Referring to FIG. 5, as the thrombus 230 is broken up, the first elongate element 110 is capable of removing the pieces of the thrombus 230 that come loose. This is accomplished by providing suction through the lumen 116 of the first elongate element 110 via a suction source 1000. Because the lines 150 have broken up the thrombus 230, the pieces of the clot are small enough to travel through the lumen 116 of the first elongate element 110. The frame 130 can further act to remove at least portions of the thrombus from the vessel walls 225 as the device 100 is pulled along the vessel walls 225. These portions are also capable of being removed through the lumen 116 of the first elongate element 110. It is understood that some portions of the clot may not be pulled into the lumen 116 of the first elongate element 110, but the frame 130 and/or membrane 170 may contain the clot during removal.

During the procedure, contrast may be provided through the second elongate element 120. The lumen 126 extends through the second elongate element 120, the contrast capable of being transported through the lumen 126 such that the contrast can be delivered through the openings 128. The contrast can be released at any time during the procedure including prior to, during, and after positioning, advancing, and deploying the device 100. The contrast may be delivered on either side of a thrombus 230, including upstream or downstream.

The frame 130, including a portion of the membrane 170, can be in contact with the inner vessel wall. Thus, if thrombotic fragments are dislodged from the thrombus 230 as a result of the displacement or cutting of the thrombotic material by the frame 130, the thromboemboli can be captured by the membrane 170. For example, liberated thromboemboli may be carried by blood via the blood-flow path distally through the frame 130 and into a space defined by the membrane 170. The blood may then pass through the membrane 170, for example through small pores in the membrane 170, while the thromboemboli may be captured or trapped within the membrane 170 because the thromboemboli may be too large to pass through the pores in the membrane 170. In this manner, dislodged thrombotic fragments can be prevented from becoming fugitive thromboemboli within the bloodstream. In some embodiments, the frame 130 may be provided such that the frame 130 retains the thromboemboli.

In the case of a neurological vascular thrombus occlusion, restoring perfusion as described above is an initial treatment pursuant to saving a patient's life. Restoring downstream perfusion, even if only partial perfusion, restores blood flow to downstream neurological tissues. Restoring blood flow may also minimize and/or eliminate the pressure of blood pushing on the thrombus 230 and the vacuum or negative pressure located just distally of the thrombus 230. The reduction or elimination of that pressure differential on the sides of the thrombus 230 can enhance the effectiveness of the device 100.

In some embodiments, the construction of the frame 130 can permit some portions of the thrombus 230 to penetrate between the lines 150 and/or the struts 132, 134, 136 to within the interior space 180 of the frame 130. As a result of opening or enlarging a blood-flow path as described above, an increased amount of blood can then flow over the surface of the thrombus 230, thereby encouraging thrombolysis of thrombus 230. That is, causing additional blood to flow over the surface of the thrombus 230 can enhance the effects from blood's natural tendency to dissolve the thrombus. The blood's lytic action may partially erode surface 232 of thrombus 230 by dissolving some of the thrombus 230, or by dislodging some thrombotic particles. Dislodged thrombotic particles can be captured in membrane 170 to prevent them from becoming thromboemboli in the bloodstream.

Referring to FIG. 7, in some embodiments, the lines 150 may be provided extending between the frame 130 and the second elongate element 120. The lines 150 may be tensioned by pulling the second elongate element 120 into the first elongate element 110 such that the first elongate element 110 deflects the lines 150. It is understood that the lines 150 may be coupled to various other parts and at various positions, including to the first elongate element 110 on the interior surface of the first elongate element 110.

These embodiments may be implemented as a reverse thrombectomy, meaning the device 100 may extend through the thrombus 230, the device 100 is deployed, and the device 100 is longitudinally translated back through the thrombus 230 to disrupt the thrombus 230.

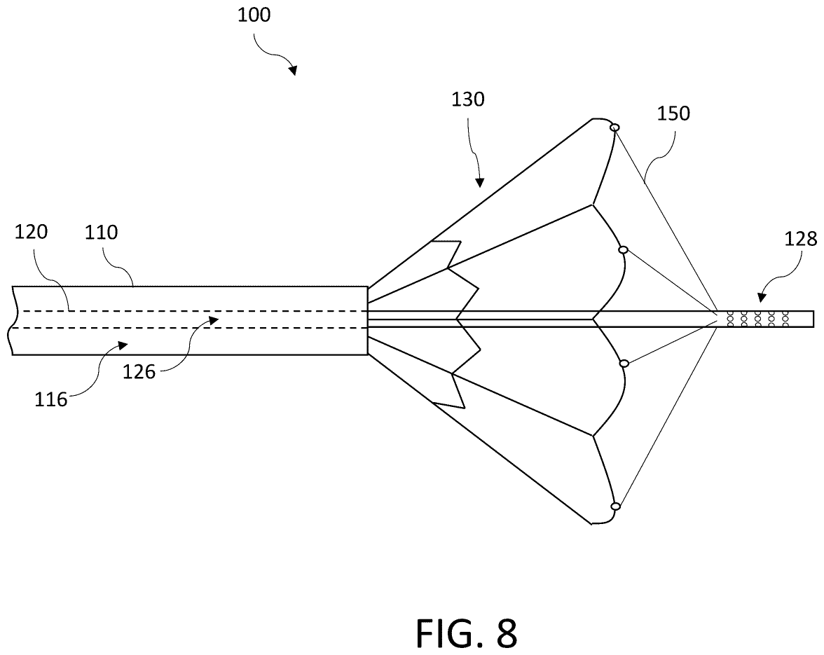

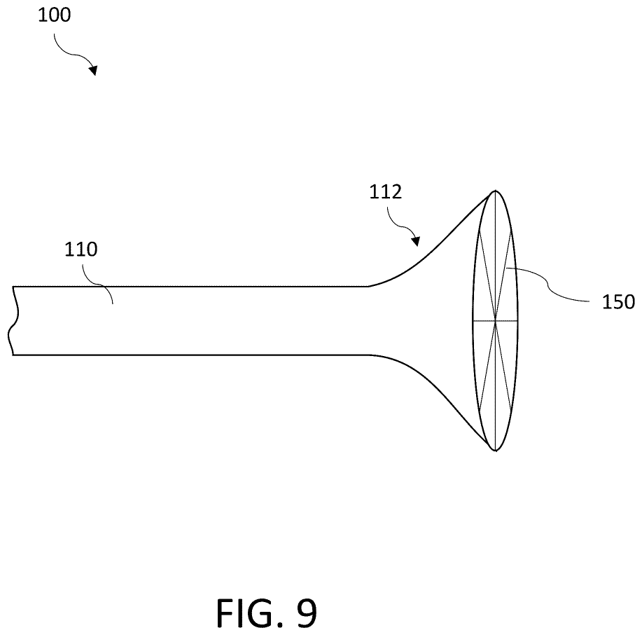

In other embodiments, the device 100 may be provided as a forward thrombectomy device. For example, FIGS. 8 and 9 depict devices including lines 150 that are positioned beyond a first elongate element 110 and a frame 130. In these embodiments, the device 100 is deployed within the vasculature of a patient and then advanced forward. As the device 100 is advanced, the lines 150 cut through the thrombus 230. The thrombus 230 is broken up and can be removed through the lumen 116 of the first elongate element 110. For example, FIG. 8 depicts a device 100 in which a frame 130 is deployed and the lines 150 are positioned extending between the frame 130 and the second elongate element 120. The frame 130 facilitates blood flow through the frame 130 during performance of the thrombectomy. Any of the features discussed herein may also be implemented, including but not limited to the membrane 170. Referring to FIG. 9, the first elongate element 110 may be tapered or fluted at the first end 112 and lines 150 are coupled across an opening of the first elongate element 110. In each of the embodiments, the lines 150 are leading when the device 100 is advanced and contact and cut the thrombus 230. Referring specifically to FIG. 9, in those embodiments in which the first end 112 is tapered or fluted, the first end 112 may be constrained to a diameter that is substantially similar to the diameter of the remainder of the first elongate element 110 (e.g., constrained by a constraining member), and when released from constrained configuration may expand (e.g., self-expand) to the larger, fluted diameter.

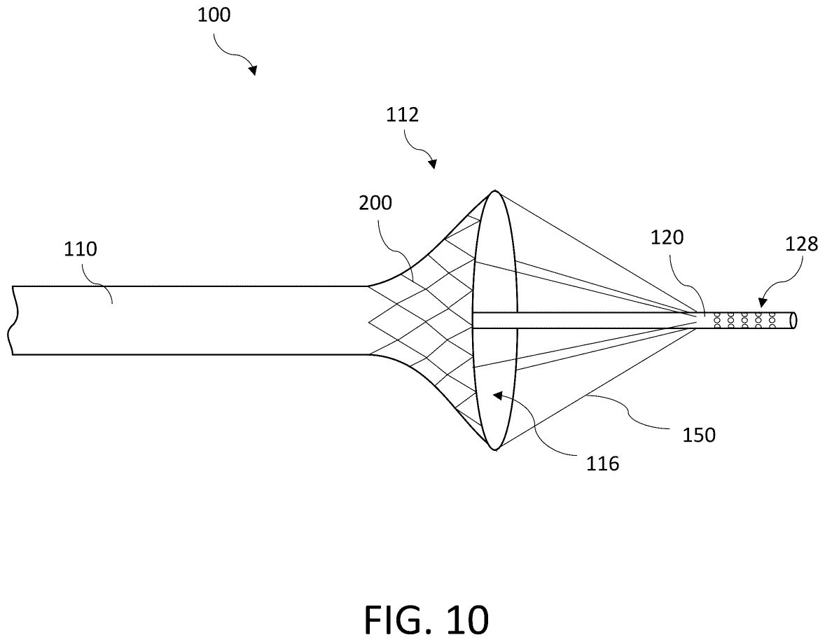

Referring to FIG. 10, the device 100 may be provided as a forward thrombectomy device with a first elongate element 110 and a second elongate element 120. The first elongate element 110 may be tapered or fluted at the first end 112. The first end 112 may also be supported by a support frame 200. The support frame 200 may be self-expanding in some embodiments. A plurality of lines 150 extend from the first end 112 of the first elongate element to the first end 122 of the second elongate element 120. The lines 150 may act as a cutter to a thrombus or clot. The second elongate element 120 may also include ports or apertures 128 as previously discussed with respect to other embodiments. The pieces of the clot or thrombus may be broken up and then received into the lumen 116 of the first elongate element 110 for removal. As discussed with respect to FIG. 9, the device 100 of FIG. 10 may likewise be constrained such that the first end 112 includes a similar diameter and then when unconstrained assumes a fluted or tapered profile. Furthermore, the fluting or taper of the first end 112 may be adjusted or collapsed by tensioning the lines 150 such that the second end is at least partially collapsed to a smaller diameter.

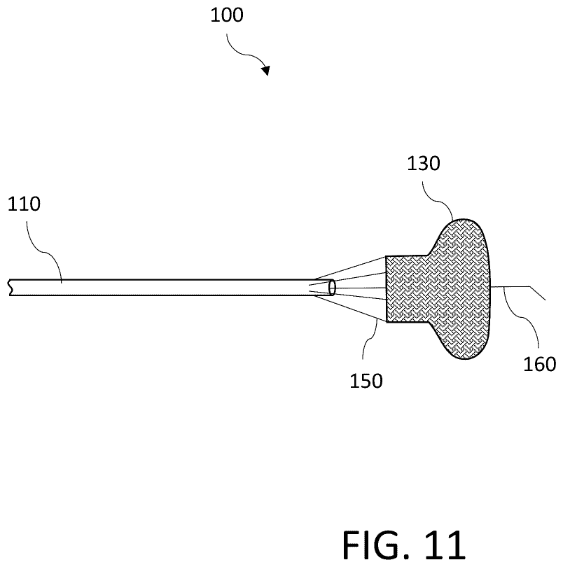

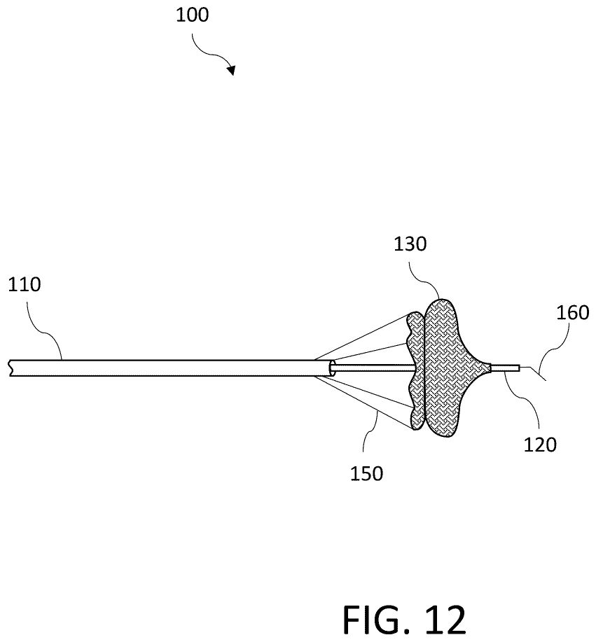

Referring to FIGS. 11 and 12, the device 10 may be provided with the frame 130 that is capable of expansion (e.g., self-expansion or otherwise). The frame 130 may be formed of a braided construct (e.g., braided nitinol, braided stainless steel, etc.). The lines 150 may be coupled to the frame 130 and the first elongate element 110 as previously described. The lines 150 may cut the thrombus or clot which may be removed through the first elongate element 110 as previously discussed. The frame 130 may be provided in a variety of shapes and configurations to suit the needs of the procedure, position, and anatomy. For example, one shape of configuration may be provided for use in removing a pulmonary embolism whereas another shape or configuration may be used in the iliac vein. In some embodiments, the frame 130 is coupled to a second elongate element 120 to control, deploy, and recapture the frame 130 during use (see FIG.

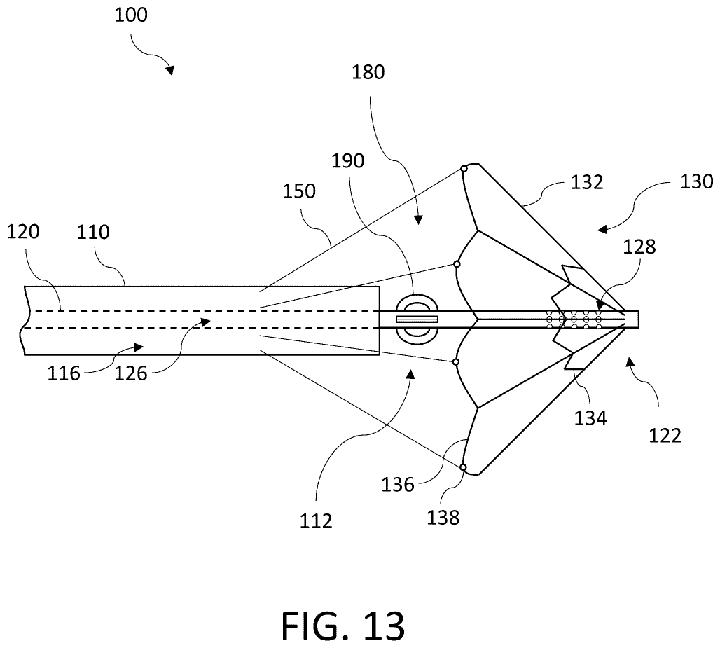

Referring to FIG. 13, the device may be provided with a secondary cutter 190. The secondary cutter 190 may be positioned proximal to the first end 112 of the first elongate element 110 such that portions of the clot that are being received into the first elongate element 110 may be further broken up or macerated in order to be accommodated within the first elongate element 110. The secondary cutter 190, for example, may be positioned on the second elongate element 120 proximal an opening of the first elongate element 110 at the first end 112. The secondary cutter 190 may, in some embodiments, include a plurality of blades spaced circumferentially about the second elongate element 120. The blades may be positioned longitudinally such that the blades break up the clot as they are received substantially longitudinally into the first elongate element 110. It is understood that various configurations including number of secondary cutters (e.g. one, two, three four, five, or more), shape of secondary cutters (straight, tapered, arcuate, and so forth), and orientation of the secondary cutters (parallel to longitudinal axis of second elongate element 120, at an angle to the second elongate element 120) is contemplated herein. The secondary cutter 190 may center the frame 130 and the second elongate element 120 within the first elongate element 110. In some embodiments, the secondary cutter 190 may also aid in centering the device 100 within the patient's vessel and the thrombus. This can aid in positioning as well as deployment and retrieval of the frame 130 during use.

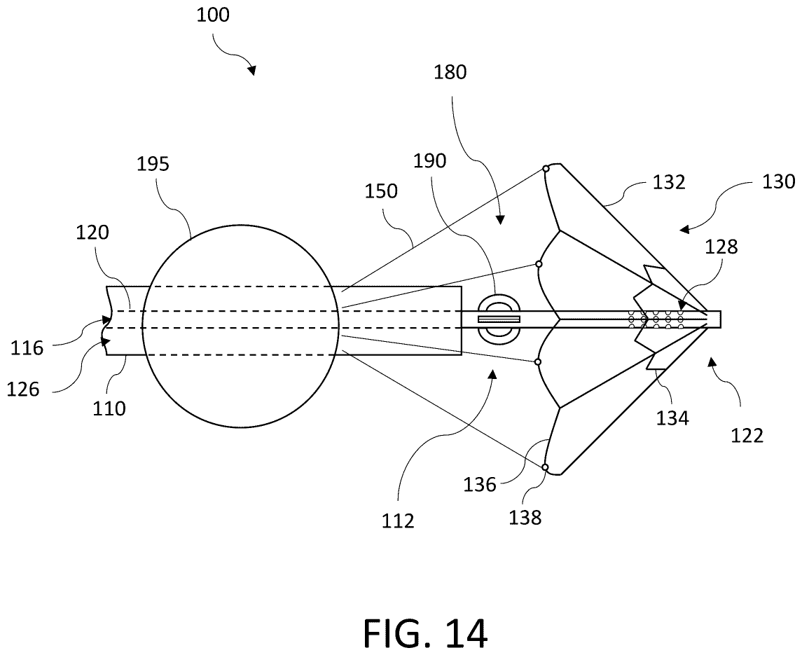

Referring to FIG. 14, in some embodiments the device 100 may include a balloon or expandable member 195, which may be positioned on the first elongate element 110. The balloon 195, for example, may be integral with the first elongate element 110. The balloon 195 may be positioned and inflated during the procedure to limit an embolism from forming as the thrombus is macerated. The balloon 195 may also limit flow during the removal of the thrombus, which may reduce the amount of suction necessary in retrograde procedures. The balloon 195 may be inflated to completely occlude the vessel or may be used to partially occlude the vessel.

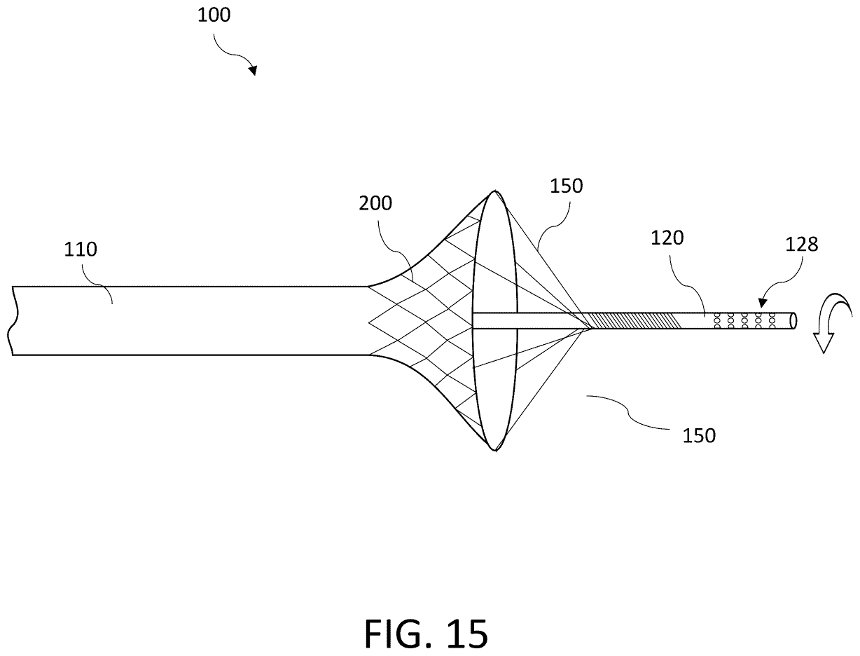

Referring to FIG. 15, the device 100 is shown in which the second elongate element 120 is rotatable or may be torqued relative to the first elongate element 110. This allows for the lines 150 to be tensioned further by wrapping about the second elongate element 120. As the lines 150 are wrapped about the second elongate element 120, the exposed portion of the lines 150 is decreased. This can allow for increased tension for cutting, and may position the lines to cut clots that are more difficult (e.g., if the clot is positioned in the interior space 180, the lines 150 may be shortened to cut into smaller portions). The torque-ability may also facilitate progressive cutting and penetration into a difficult clot. It is understood that any of the embodiments shown or discussed herein with the first and second elongate elements 110, 120 may implement this rotational configuration, and the disclosure is not limited to the embodiment of FIG. 15.

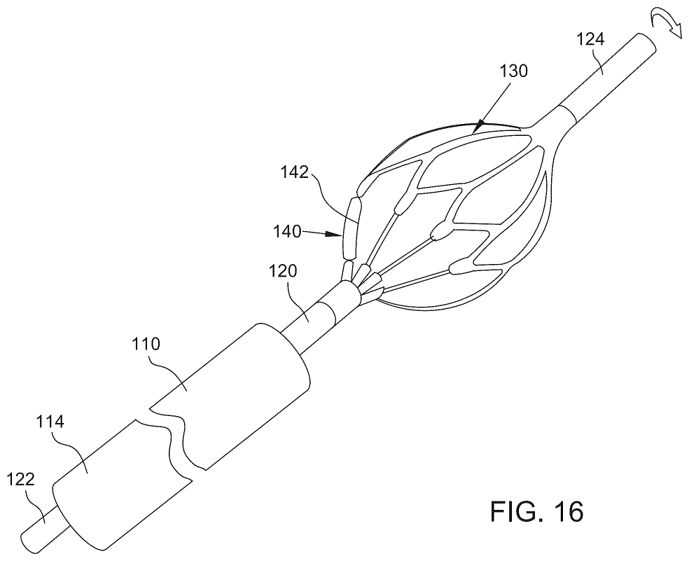

Referring to FIG. 16, the frame 130 may be formed out of a cut nitinol tube. The frame 130 may include cutting struts 140 that are capable of cutting the thrombus either through axial movement or rotational movement of the frame 130 when in contact with the thrombus. For example, the frame 130 may be cut such that the cutting struts 140 are oriented to cut the thrombus when a user pushes or pulls on the device 10. The profile of the cutting struts 140 may be optimized for cutting through the thrombus. The device 100 illustrated in FIG. 16 may be implemented in both retrograde and/or antegrade thrombus removal procedures.

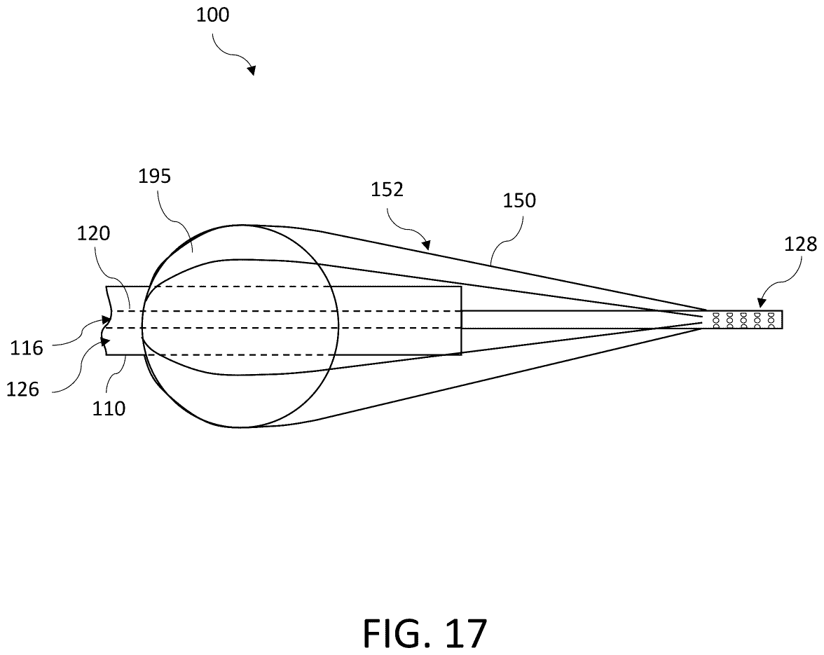

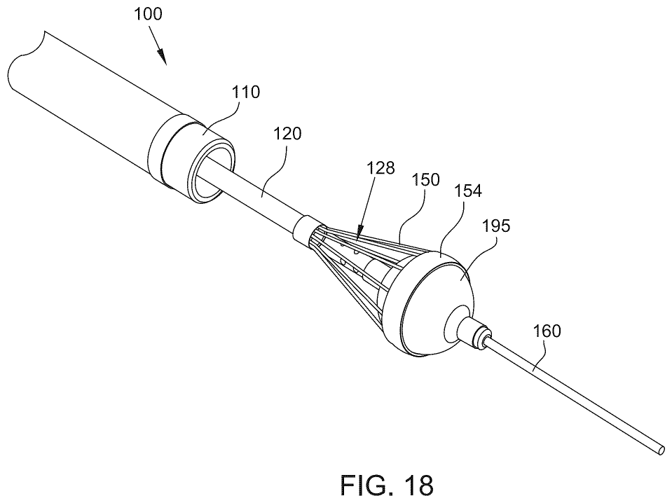

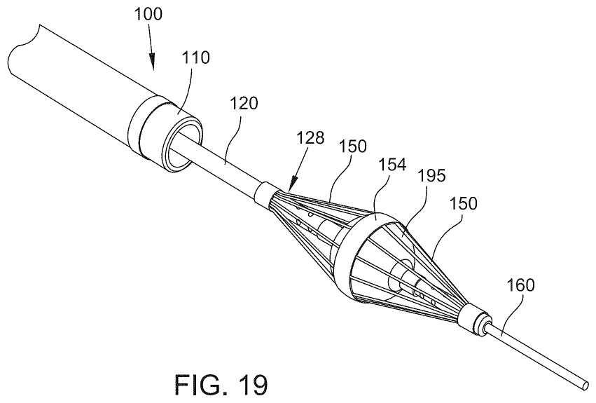

Referring to FIG. 17, in one embodiment, the device 100 is provided with a balloon 195 which is positioned on the first end 112 of the first elongate element 110. The balloon 195 is in contact with the lines 150 such that when the balloon 195 is inflated, the lines 150 are repositioned in a deployed configuration. The lines 150, for example, may extend about an exterior surface of the balloon 195 such that they are both positioned radially further (e.g. spaced) from the first and second elongate elements 110, 120 along a central portion 152 of the lines 150 when in the deployed configuration, and such that the lines 150 are tensioned. The lines 150 may further be tensioned via longitudinal movement of the second elongate element 120 relative to the first elongate element 110 and/or rotation of the second elongate element 120 relative to the first elongate element 110, as previously disclosed. The device 100 may be provided for either retrograde and/or antegrade procedures. For example, FIG. 17 illustrates a device 100 that may be implemented in an antegrade configuration, FIG. 18 illustrates a device 100 that may be implemented in a retrograde configuration, and FIG. 19 illustrates a device that may be implemented in both an antegrade and a retrograde configuration. The balloon 195 and the lines 150 may be provided on various components and at various positions. For example, FIG. 17 illustrates the balloon positioned about the first elongate element and FIGS. 18 and 19 illustrate the balloon 195 position about the second elongate element 120. The balloon 195 may be positioned proximal or distal the apertures 128 that may be implemented for providing contrast. The lines 150 may be coupled to the first elongate element 110 and the second elongate element 120 (see FIG. 17), or may be coupled to the second elongate element 120 (see FIGS. 18 and 19). It is understood that the various features described with respect to other embodiments may be implemented on the embodiments of FIGS. 17-20, including but not limited to the secondary cutter 190, rotation for tensioning, contrast, braided structures, and so forth. As illustrated in FIGS. 18 and 19, a ring 154 may be implemented to limit contact between the balloon 195 and the lines 150, thus decreasing the possibility of the lines 150 cutting the balloon 195. The lines 150 may be coupled to the ring, or may be positioned against the ring 154 to limit contact. The ring 154 may be expandable and collapsible with the balloon 195, or may include a constant diameter.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

1. A medical device for treating one of a thrombus and embolism, comprising:
a first elongate element having a first end and a second end, the first elongate element defining a lumen and having a first diameter;
a second elongate element extending through at least a portion of the lumen of the first elongate element, the second elongate element having a first end and a second end, wherein the first end of the second elongate element is configured to extend beyond the first end of the first elongate element;
a frame extending from the first end of the second elongate element, the frame having a second diameter when in a deployed configuration, wherein the second diameter is greater than the first diameter of the first elongate element; and
a plurality of lines coupled to the first elongate element and the frame, the plurality of lines operable to be tensioned when the frame is in the deployed configuration, the plurality of lines operable to cut through at least one of an embolism or thrombus.

2. (canceled)

3. (canceled)

4. The medical device of claim 1, wherein the second elongate element is longitudinally moveable relative to the first elongate element.

5. (canceled)

6. The medical device of claim 5, wherein the second elongate element includes at least one opening proximate the first end, the second elongate element is operable to be fluidically coupled to a fluid source including at least one of a contrast source, a saline source, and a therapeutic source.

7. (canceled)

8. The medical device of claim 1, wherein the frame includes a porous covering.

9. The medical device of claim 1, wherein the frame defines an outer perimeter, wherein each line of the plurality of lines is coupled to the frame proximate the outer perimeter.

10. The medical device of claim 9, wherein the frame includes a plurality of radial struts extending from the second elongate element toward the outer perimeter of the frame.

11. The medical device of claim 10, wherein each line of the plurality of lines is coupled to the frame at the plurality of radial struts.

12. The medical device of claim 9, wherein the frame includes a plurality of peripheral struts extending between the plurality of radial struts proximate the outer perimeter of the frame, wherein the plurality of lines are coupled to the frame at the plurality of peripheral struts between the plurality of radial struts.

13. The medical device of claim 1, wherein each line of the plurality of lines includes an ePTFE filament.

14. The medical device of claim 1, wherein each line of the plurality of lines is a braided fiber.

15. The medical device of claim 1, wherein the plurality of lines is coupled to an outer surface of the first elongate element.

16. The medical device of claim 1, further comprising a secondary cutter coupled to the second elongate element proximate the first end of the first elongate element.

17. The medical device of claim 1, further comprising an expandable member coupled to the first elongate element.

18. The medical device of claim 17, wherein the expandable member is positioned interior to the plurality of lines such that when the expandable member is expanded, the plurality of lines are tensioned.

19. The medical device of claim 1, wherein the second elongate element is rotatable relative to the first elongate element, wherein the plurality of lines are operable to be tensioned when the second elongate element is rotated.

20. The medical device of claim 1, wherein the plurality of lines is coupled to an outer surface of the second elongate element.

21. (canceled)

22. (canceled)

23. (canceled)

24. (canceled)

25. A medical device for treating at least one of a thrombus and embolism, comprising:
an elongate element having a first end and a second end, the first elongate element defining a lumen and having a first diameter, the first end including an opening to the lumen; and
a plurality of lines coupled to the first elongate element such that the lines are extending across the opening, the plurality of lines operable to be tensioned when the elongate element is in a deployed configuration, the plurality of lines operable to cut through at least one of an embolism or thrombus.

26. (canceled)

27. (canceled)

28. (canceled)

29. (canceled)
